(19) United States
(12) Patent Application Publication
(10) Pub. No.: US 2008/0319103 A1
(43) Pub. Date: Dec. 25, 2008

(54) Polyurethane composite material and application

(75) Inventors: Kuo-Huang Hsieh, Taipei City (TW); Chun-Pin Lin, Taipei City (TW); Ken-Hsuan Liao, Taipei City (TW); Chung-Yi Lee, Taipei City (TW)

Correspondence Address: WPAT, PC, 7225 BEVERLY ST., ANNANDALE, VA, 22003, US

(73) Assignee: NATIONAL TAIWAN UNIVERSITY, Taipei City (TW)

(21) Appl. No.: 11/765,575

(22) Filed: Jun. 20, 2007

(51) Int. Cl. A61K 6/09

(52) U.S. Cl. 523/116

(57) ABSTRACT

The present invention discloses a Polyurethane (PU) composite material comprising a thermoplastic polyurethane and a filler. The thermoplastic polyurethane comprises a linear polyurethane main-chain, and the polyurethane main-chain comprises a soft segment and a hard segment. The hard segment of the polyurethane main-chain is formed by a diisocyanate and a chain extender, and the soft segment of the polyurethane main-chain is formed by a polyol. The present invention also teaches the application of the disclosed thermoplastic polyurethane composite material in dental root canal material.

### BACKGROUND OF THE INVENTION

1. Field of the Invention

The present invention is generally related to a composite material, and more particularly to a thermoplastic polyurethane(TPU) material and application thereof in endodontic obturation.

2. Description of the Prior Art

The purpose of root canal treatment is to create an end result where the tissues that surround a tooth's root will maintain a healthy status despite the fact that the tooth's nerve has undergone degenerative changes. Root canal treatment occurs in three stages: First comes the diagnosis. Next comes the root canal itself, in which a dentist or an endodentist (a dentist who specializes in treating the inside of the tooth) removes the pulp (and thereby the infection), and cleans the inside of the tooth preparatory to filling it, sometimes applying antibiotics to thwart further infection. A temporary filling is placed at the crown opening. Finally, in a subsequent appointment, a crown is installed to seal the tooth and protect it from further damage or infection.

Root canal obturation involves inserting a filling cone into a root canal and cementing it therein to obturate the canal using a sealer. The common root canal filling cone material is made from gutta-percha or resilon. Lateral condensation and vertical condensation of warm or hot gutta-percha/resilon are methods be used in sealing root canals. After cementing a primary cone short of apex of the root canal, heat application is alternated with a series of smaller and smaller pluggers until the gutta-percha or resilon is moved to the apex.

The traditional root canal material is inert in nature and will not be absorbed or degraded by living tissue if the root canal is overfilled and extends beyond the apex. It has been a challenge for dentists to control the exact amount of the material within the border of the root canal to avoid overfilling. The cold core of the root canal material is not malleable so that it cannot be molded to the canal walls, resulting in poor adherence. In addition, when heated the root canal material cools to body temperature in the root, a uniform contraction takes place further reducing adherence to the root canal walls. Moreover, the filling is a polyisoprene rubber material in nature, which does not have the capability to bond to most dental materials, especially when the root canal sealer is a polymer-based material. Due to poor adherence and bonding, bacteria residential in the root canal can multiply or a leakage may result, causing bateria to enter the canal from the mouth, which can lead to the persistence of an infection or other complication. According to the above, it is important to develop a novel set of root canal material which has high biocompatibility, low volume contractive rate, better chemical-bonding ability with dentinal wall and the filling and high mechanical properties.

### SUMMARY OF THE INVENTION

In light of the above background, the present invention provides a Polyurethane composite material and application thereof.

One object of the present invention is to provide a thermoplastic polyurethane composite material. By changing the molar composition of the components such as polyol and diisocyanate, physical and chemical property of the disclosed thermoplastic polyurethane composite material can be adjusted.

Another object of the present invention is to apply the thermoplastic polyurethane composite material as the cone material in endodontic treatment. The formed cone material is various in physical and chemical properties by adjusting the molar ratio of polyol and diisocyanate, which are the forming components of the disclosed thermoplastic polyurethane composite material. Moreover, the polyurethane composite material can mix with an antibiotic material to increase application performance. According to the above, the present invention does have the economic advantages for industrial applications.

Accordingly, the present invention discloses a Polyurethane (PU) composite material comprising a thermoplastic polyurethane and a filler. The thermoplastic polyurethane comprises a linear polyurethane main-chain, which comprises a soft segment and a hard segment, wherein the hard segment is formed by a diisocyanate and a chain extender, and the soft segment is formed by a polyol. In addition, the disclosed thermoplastic polyurethane composite material can be applied as dental root canal material.

### DESCRIPTION OF THE PREFERRED EMBODIMENTS

What is probed into the invention is a polyurethane composite material and application thereof. Detail descriptions of the structure and elements will be provided in the following in order to make the invention thoroughly understood. Obviously, the application of the invention is not confined to specific details familiar to those who are skilled in the art. On the other hand, the common structures and elements that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

The first embodiment of the present invention discloses a polyurethane composite material, comprising a thermoplastic polyurethane and mixed with a filler. The above-mentioned thermoplastic polyurethane comprises a linear polyurethane main-chain, the polyurethane main-chain more comprises a soft segment and a hard segment. The hard segment of polyurethane main-chain is formed by a diisocyanate and a chain extender. The soft segment of polyurethane main-chain is formed by a polyol. In addition, the above-mentioned filler is inorganic material and better comprises zinc oxide (ZnO) fluoroaluminosilicate glass, etc. The chain extender can be 1,4-Butanediol(1,4-BD).

The above-mentioned diisocyanate can be aromatic polyisocyanates, alicyclic polyisocyanates or aliphatic polyisocyanates. In detail, the diisocyanate can be selected from the group consisting of the following: (1) aromatic polyisocyanates: tolylene diisocyanate (TDI)(2,4- or 2,6-TDI), diphenylmethane diisocyanate (MDI)(4,4′- or 2,4′-MDI), polymeric MDI, xylylene diisocyanate (XDI), naphthylene diisocyanate (NDI)(usually 1,5-NDI), paraphenylene diisocyanate (PPDI), tetramethylxylylene diisocyanate (TMXDI), tolidine diisocyanate (TODI), 3,3′-dimethoxy-4,4′-biphenylene diisocyanate, etc. (2) alicyclic polyisocyanates: dicyclohexylmethane diisocyanate (HMDI)(4,4′- or 2,4′-HMDI), isophorone diisocyanate (IPDI), isopropylidene-bis-(4-cyclohexylisocyanate) (IPC), hydrogenated xylylene diisocyanate (hydrogenated XDI), cyclohexylene diisocyanate (CHPI) (usually 1,4-CHPI), 1,5-tetrahydonaphthalene diisocyanate, etc. (3) aliphatic polyisocyanates: hexamethylene diisocyanate (HDI), lysine diisocyanate (LDI), tetramethylene diisocyanate, etc. Better practices, the diisocyanate comprises one selected from the group consisting of the following: alicyclic polyisocyanates and aliphatic polyisocyanates.

The above-mentioned polyol can be poly-ester polyol or poly-ether polyol. In detail, the polyol can be selected from the group consisting of the following: (1) poly-ester polyol: Polycaprolactone (PCL), Poly(butylene-adipate) glycol (PBA), Poly(ethylene-adipate) glycol (PEA), Poly(ethylene-butylene-adipate) glycol (PEBA), Poly(hexylene-adipate) glycol (PHA); (2) poly-ether polyol: Polyethylene Glycol (PEG), Polypropylene Glycol (PPG), Polytetramethylene Glycol (PTMEG), Polytetramethylene Oxide (PTMO).

### EXAMPLE 1

### Thermal Properties of Polyurethane Composite Material

This example compares the impact of various diisocynanates and polyol/diisocynanate/chainextender weight compositions on the thermal properties of formed polyurethane composite material. For each sample, the weight ratio of thermoplastic polyurethane and filler is less than 0.5. In this example, the polyol is selected to be poly (butylene-adipate) glycol (PBA) and the chain extender is 1,4-Butanediol(1,4-BD). The result of comparison is shown in Table 1.

TABLE 1
Thermal properties of polyurethane composite material

| poly-ol/diisocyanate/ | | |
|---|---|---|
| chain extender | | |
| (TPU: 30 wt % | Melting point (° C.) | |
| of total composite) | HDI as diisocyanate | IPDI as diisocyanate |
| 1/1.60/0.5 | 54.1 (Composite H1.) | 48.6 (Composite IP1.) |
| 1/1.36/0.3 | 53.9 (Composite H2.) | 48.0 (Composite IP2.) |
| 1/1.12/0.1 | 54.1 (Composite H3.) | 48.3 (Composite IP3.) |

(TPU: 30 wt % of total composite)

It is noted that the melting point of commercial gutta-percha is about 60.01° C., and the melting point of commercial resilon is about 60.01° C. (reference: A comparison of thermal properties between gutta-percha and a synthetic polymer based root canal filling material (Resilon). Journal of Endodontics, 2006 Jul; 32(7):683-6. Miner M R, Berzins D W, Bahcall J K.)

As shown in the result, when the weight ratio of polyol and diisocynanate is equal to 0.8, the sample utilizing Hexamethylene Diisocyanate (HDI) as diisocyanate to form polyurethane composite material has a melting point lower than 55° C. On the other hand, the sample utilizing Isophorone Diisocyanate(IPDI) as diisocyanate to form polyurethane composite material has a melting point lower than 48.6° C. There is no significant change on the melting point of the polyurethane composite material when the weight ratio of polyol and diisocynanate increases (higher than 0.8). In both the HDI system and IPDI system, the melting point is lower than 55° C., which is also lower than that of commercial gutta-percha and resilon. In clinical root canal treatment, dentists usually have to conduct material into the root canal. For this purpose, the cone material is usually heated into liquid condition in advance. Therefore, the melting point of the cone materials cannot to too high. Accordingly, the present invention does have the economic advantages for industrial applications.

### EXAMPLE 2

### Mechanical Properties of Polyurethane Composite Material

This example compares the impact of various polyol/diisocynanate/chainextender weight ratio on the thermal properties of formed polyurethane composite material. The weight ratios of the thermoplastic polyurethane and filler are lower than 0.5. In this example, the polyol is selected to be poly (butylene-adipate) glycol(PBA) and the chain extender is 1,4-Butanediol(1,4-BD). The result of comparison is shown in Table 2.

TABLE 2
Mechanical properties of polyurethane composite material

| | TPU | | |
|---|---|---|---|
| | Composition | | |
| | (Polyol/ | | |
| | Diisocyanate/ | Mechanical Properties | |
| | Chain | Tensile Strength | Yang's Modulus |
| Designations | extender) | (Mpa) | (Mpa) |
| Composite H1 | 1/1.60/0.5 | Not-available | Not-available |
| Composite H2 | 1/1.36/0.3 | Not-available | Not-available |
| Composite H3 | 1/1.12/0.1 | 21.8 ± 2.6 | 130.0 ± 18.3 |
| Composite IP1 | 1/1.60/0.5 | Not-available | Not-available |
| Composite IP2 | 1/1.36/0.3 | 0.8 ± 0.3 | 32.1 ± 13.4 |
| Composite IP3 | 1/1.12/0.1 | 15.8 ± 1.9 | 96.2 ± 17.7 |
| Gutta-percha | — | 5.98 ± 1.15 (a) | 78.71 ± 23.41 (a) |
| Resilon | — | 8.09 ± 2.30 (a) | 86.58 ± 42.23 (a) |

As shown in the table, tensile strength of commercial gutta-percha and resilon is generally less than 10 MPa, and Young's Modulus of gutta-percha and resilon is generally less than 90 MPa. In this example, when the weight ratio of polyol and diisocynanate is greater than 0.88 (polyol: diisocynanate=1:1.12), the sample utilizing HDI as diisocyanate to form polyurethane composite material has a tensile strength and Young's Modulus of 10 MPa (IP3; 15.8 MPa) and 90 MPa (IP3; 96.2 MPa), respectively. The sample utilizing IPDI as diisocyanate to form polyurethane composite material has a tensile strength and Young's Modulus of 20 MPa (H3; 21.8 MPa) and 100 MPa (H3; 130.0 MPa), respectively. The mechanical properties of root canal material determine the post-treatment tooth tightness and chewing ability, therefore the present invention has a great potential in the application of root canal material.

The second embodiment of the present invention discloses a cone material in root canal material. The above-mentioned cone material comprises a polyurethane composite material described in the first embodiment, and can further mix with an antibiotic material to increase its performance.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A polyurethane composite material, comprising:
a thermoplastic polyurethane, wherein the thermoplastic polyurethane comprises a linear polyurethane main-chain, the polyurethane main-chain comprises a soft segment and a hard segment, the hard segment is formed by a diisocyanate and a chain extender, the soft segment is formed by a polyol, the polyol comprises one selected from the group consisting of the following: poly-ester polyol and poly-ether polyol; and
a filler.

2. The polyurethane composite material according to claim 1, wherein the diisocyanate comprises one selected from the group consisting of the following: (1) aromatic polyisocyanates: tolylene diisocyanate (TDI)(2,4- or 2,6-TDI), diphenylmethane diisocyanate (MDI)(4,4′- or 2,4′-MDI), polymeric MDI, xylylene diisocyanate (XDI), naphthylene diisocyanate (NDI)(usually 1,5-NDI), paraphenylene diisocyanate (PPDI), tetramethylxylylene diisocyanate (TMXDI), tolidine diisocyanate (TODI), 3,3′-dimethoxy-4,4′-biphenylene diisocyanate; (2) alicyclic polyisocyanates: dicyclohexylmethane diisocyanate (HMDI)(4,4′- or 2,4′-HMDI), isophorone diisocyanate (IPDI), isopropylidene-bis-(4-cyclohexylisocyanate) (IPC), hydrogenated xylylene diisocyanate (hydrogenated XDI), cyclohexylene diisocyanate (CHPI)(usually 1,4-CHPI), 1,5-tetrahydonaphthalene diisocyanate; (3) aliphatic polyisocyanates: hexamethylene diisocyanate (HDI), lysine diisocyanate (LDI), tetramethylene diisocyanate.

3. The polyurethane composite material according to claim 1, wherein the diisocyanate comprises one selected from the group consisting of the following: alicyclic polyisocyanates and aliphatic polyisocyanates.

4. The polyurethane composite material according to claim 1, wherein the diisocyanate is hexamethylene diisocyanate (HDI).

5. The polyurethane composite material according to claim 1, wherein the poly-ester polyol is selected from the group consisting of the following: Polycaprolactone (PCL), Poly(butylene-adipate) glycol (PBA), Poly(ethylene-adipate) glycol (PEA), Poly(ethylene-butylene-adipate) glycol (PEBA), and Poly(hexylene-adipate) glycol (PHA).

6. The polyurethane composite material according to claim 1, wherein the poly-ether polyol is selected from the group consisting of the following: Polyethylene Glycol (PEG), Polypropylene Glycol (PPG), Polytetramethylene Glycol (PTMEG), and Polytetramethylene Oxide (PTMO).

7. The polyurethane composite material according to claim 1, wherein the polyol is Poly(butylene-adipate) glycol (PBA).

8. The polyurethane composite material according to claim 1, wherein the filler is selected from the group consisting of the following: zinc oxide (ZnO), fluoroaluminosilicate glass.

9. The polyurethane composite material according to claim 1, wherein the weight ratio of the polyol and the diisocyanate is equal to or greater than 0.8.

10. The polyurethane composite material according to claim 1, wherein the weight ratio of the thermoplastic polyurethane and the filler is equal to or less than 0.5.

11. The polyurethane composite material according to claim 1, wherein the melting point of the polyurethane composite material is equal to or lower than 60° C.

12. The polyurethane composite material according to claim 1, wherein the melting point of the polyurethane composite material is equal to or lower than 55° C.

13. The polyurethane composite material according to claim 1, wherein the tensile strength of the polyurethane composite material is equal to or greater than 10 MPa.

14. The polyurethane composite material according to claim 1, wherein the tensile strength of the polyurethane composite material is equal to or greater than 20 MPa.

15. The polyurethane composite material according to claim 1, wherein the Young's Modulus of the polyurethane composite material is equal to or greater than 90 MPa.

16. The polyurethane composite material according to claim 1, wherein the Young's Modulus of the polyurethane composite material is equal to or greater than 100 MPa.

17. A cone material in root canal material, wherein the cone material comprises a polyurethane composite material as described in claim 1.

18. The cone material according to claim 17, wherein the cone material is mixed with an antibiotic material.
